(19) United States
(12) Patent Application Publication
(10) Pub. No.: US 2024/0095805 A1
(43) Pub. Date: Mar. 21, 2024

(54) RECOMMENDER SYSTEM FOR TIME-DEPENDENT ACTIONS BASED ON TEMPORAL KNOWLEDGE GRAPHS

(71) Applicant: NEC Laboratories Europe GmbH, Heidelberg (DE)

(72) Inventors: Timo SZTYLER, Heidelberg (DE); Julia GASTINGER, Heidelberg (DE)

(21) Appl. No.: 18/275,073

(22) PCT Filed: Feb. 24, 2021
(86) PCT No.: PCT/EP2021/054592

§ 371 Date: Jul. 31, 2023

(30) Foreign Application Priority Data
Feb. 5, 2021 (EP) 21155554.5

(51) Int. Cl. G06Q 30/0601

(52) U.S. Cl. CPC G06Q 30/0631

(57) ABSTRACT

A method for providing recommendations to users based on a state of interest is provided. The method includes organizing domain of interest information in an initial temporal knowledge graph, wherein t is a timestamp that refers to a present point in time. The method predicts, for at least one future point in time (t+x), future entities, future links between entities and/or future attributes of entities for the initial knowledge graph and produces at least one new knowledge graph based on the predictions, simulates situations resulting from the execution of a particular action or a combination of actions at certain points in time and predicting expected temporal knowledge graphs for the simulated situations, and classifies the knowledge graphs produced for the respective points in time and for the simulated situations based on the state of interest. A ranked list of recommended actions is provided based on the classification result.

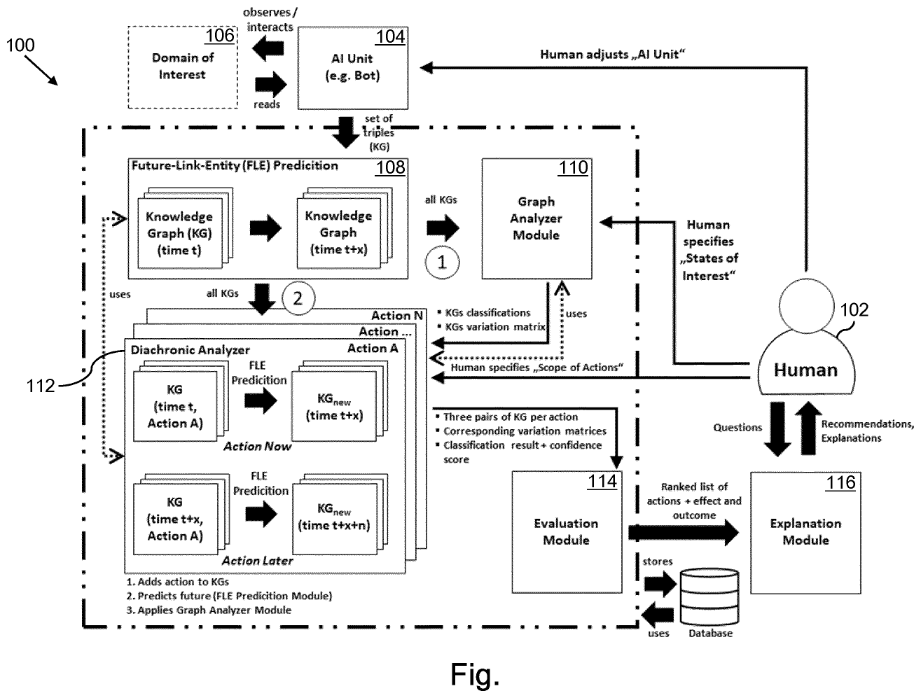

### CROSS REFERENCE TO RELATED APPLICATIONS

This application is a U.S. National Phase application under 35 U.S.C. § 371 of International Application No. PCT/EP2021/054592, filed on Feb. 24, 2021, and claims benefit to European Patent Application No. EP 21155554.5, filed on Feb. 5, 2021. The International Application was published in English on Aug. 11, 2022 as WO 2022/167103 A1 under PCT Article 21(2).

### FIELD

The present invention relates to a method and a recommender system for providing recommendations to users based on a state of interest.

### BACKGROUND

In recent years, several improvements have been achieved with respect to recommender systems (RS) that assist users in making accurate selections from a big amount of available product or services. In particular, the effectiveness of knowledge graphs (KG) in providing external knowledge to further improve such systems has been demonstrated.

However, where existing knowledge graph-based recommender systems are able to deal with a big amount of relational data, as well as multi-modality and multi-dimensionality, they cannot consider the time aspect. This shortcoming is twofold:

First, Dynamicity is still an open research area (for reference, see Gao, Yang, Yi-Fan Li, Yu Lin, Hang Gao, and Latifur Khan. “Deep Learning on Knowledge Graph for Recommender System: A Survey.” arXiv preprint arXiv:2004.00387 (2020). Dynamicity means the fact that nodes and edges may appear or disappear and types of relations between nodes may change over time. Only few knowledge-graph based systems consider partially accommodating their frameworks to cases where KG contains dynamic relations (for reference, see Song, Weiping, Zhiping Xiao, Yifan Wang, Laurent Charlin, Ming Zhang, and Jian Tang. “Session-based social recommendation via dynamic graph attention networks.” In Proceedings of the Twelfth ACM International Conference on Web Search and Data Mining, pp. 555-563. 2019).

Second, existing graph-based recommender systems do not consider the time aspect of possible actions. It is considered which action might be best for a given situation (e.g. which item to recommend to a user), but it is not considered when to best give this recommendation (e.g. now, in +1 time step, in +2 time steps, . . . ) or when in the future this will lead to certain situations. This leads to loss of information.

When trying to solve the above issues with currently available resources, the following problems arise: On the one hand, humans suffer from information overload, since humans cannot process such a big amount of data (that is typically involved in such systems), including the relations between entities and the multi-dimensionality aspect. Thus, humans are not capable of reliably predicting the outcome of possible actions for given embodiments. On the other hand, AI-based recommender systems (non-graph-based) cannot take into account the relations between the entities, the multiple dimensions and the multi-modal data and are thus not able to incorporate all available information.

### SUMMARY

In an embodiment, the present disclosure provides a method for providing recommendations to users based on a state of interest, the method comprising: organizing domain of interest information in an initial temporal knowledge graph (KGₜ), wherein t is a timestamp that refers to a present point in time; predicting, for at least one future point in time (t+x), future entities, future links between entities and/or future attributes of entities for the initial knowledge graph (KGₜ) and producing at least one new knowledge graph (KGₜ₊ₓ) based on the predictions; simulating situations resulting from the execution of a particular action or a combination of actions at certain points in time and predicting expected temporal knowledge graphs for the simulated situations; classifying the knowledge graphs produced for the respective points in time and for the simulated situations based on the state of interest; and providing, based on the classification result, a ranked list of recommended actions.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

The FIGURE illustrates an overview system architecture for deploying a recommender system according to an embodiment of the invention.

### DETAILED DESCRIPTION

In accordance with an embodiment, the present invention improves and further develops a method and a system of the initially described type for providing recommendations to users in such a way that the dynamicity is improved and that the time aspect of taking possible actions is taken into account.

In accordance with another embodiment, the present invention provides a method for providing recommendations to users based on a state of interest, the method comprising organizing domain of interest information in an initial temporal Knowledge Graph KGₜ, where t is a timestamp that refers to the present point in time; predicting, for at least one future point in time t+x, future entities, future links between entities and/or future attributes of entities for the initial Knowledge Graph KGₜ and producing at least one new Knowledge Graph KGₜ₊ₓ based on the predictions; simulating situations resulting from the execution of a particular action or a combination of actions at certain points in time and predicting expected temporal Knowledge Graphs for the simulated situations; classifying the Knowledge Graphs produced for the respective points in time and for the simulated situations based on the state of interest; and providing, based on the classification result, a ranked list of recommended actions.

Furthermore, in accordance with an embodiment, the present invention provides a recommender system for providing recommendations to users based on a state of interest, the system comprising an AI unit configured to organize domain of interest information in an initial temporal Knowledge Graph KGₜ, where t is a timestamp that refers to the present point in time; a prediction component configured to predict, for at least one future point in time t+x, future entities, future links between entities and/or future attributes of entities for the initial Knowledge Graph KGₜ and to produce at least one new Knowledge Graph KGₜ₊ₓ based on the predictions; a diachronic analyzer configured to simulate situations resulting from the execution of a particular action or a combination of actions at certain points in time and to predict expected temporal Knowledge Graphs for the simulated situations; a graph analyzer configured to classify the Knowledge Graphs produced for the respective points in time and for the simulated situations based on the state of interest; and evaluation module configured to provide, based on the classification result, a ranked list of recommended actions.

Embodiments of the present invention provide a recommender system for finding and recommending time-dependent actions based on Temporal Knowledge Graphs, TKGs, with the goal of steering members of a domain of interest towards a certain direction. The recommender system may be configured to (continuously) collect and/or acquire information from the domain of interest through interaction and observation. Furthermore, the system may comprise computer-implemented tools that are configured to analyze the information and to predict future events and developments.

According to some embodiments, the system may be configured to compute alternative futures, based on a current situation, by simulating the execution of one or more actions at a number of specific times in the future and by predicting the respective outcome. The system may comprise computer-implemented tools that are configured to compare the predicted future with the alternative futures and, based on such comparison, to detect causes for certain events and to compute proposals for actions to steer the domain of interest in a certain direction.

According to embodiments, the system provides a ranked list of those recommended actions and expected outcomes, possibly together with explanations on the actions/outcomes, to a user of the recommender system. According to embodiments, the ranked list of recommended actions may be provided together with their recommended time of execution. Based on the recommended actions (and possibly their recommended time of execution), the user can then instruct the system to take next steps. According to embodiments, it may be provided that the system is able to take user feedback into account by updating its decision-making strategy.

Embodiments of the present invention provide the following technical improvements compared to existing technologies:
1. Predicting the future development of a situation through Temporal Knowledge Graphs for different scenarios by simulating actions at a specific point in time and evaluating the outcome with Graph classification. A differentiator to classical recommender systems is that the recommender system according to embodiments of the invention has the capability to take into account time together with multi-modal and multidimensional data, i.e., the system can capture and understand complex issues and situations.
2. Considering of not only one time step, but multiple future time steps, and the fact that one or more actions applied at different time steps can lead to different outcomes.
3.1 Recommending actions not only based on the graph classification result, but also taking into account the model uncertainty of the classification result with a confidence score.
3.2 Determining a difference to alternative scenarios with variation matrices, and the cost of each action.

According to an embodiment, the prediction component, starting from an initial Knowledge Graph KGₜ representing a situation at a present time t, predicts future entities, future links between entities and/or future attributes of entities for at least one future point in time t+x, and produces for each of these future point in times t+x a respective new Knowledge Graph KGₜ₊ₓ. For performing these tasks, the prediction component may include a neural network, wherein the weights of the neural network may be trained with stochastic gradient descent, SGD, using past prediction results as training data.

According to an embodiment, the graph analyzer may be configured to characterize present and expected situations by analyzing the temporal Knowledge Graphs for the different points in time. For performing this tasks, the graph analyzer may include a neural network that—given i) a set of already classified pairs (present and future) of TKGs as training data, ii) States of Interest defined by a human (e.g., expressed in form of a set of classification labels), and iii) the TKG which needs to be classified—calculates as outcome a confidence score for each State of Interest and a weight matrix that reflects the difference between the present TKG and the respective future TKG. By means of classifying the TKGs (for each considered time-step), the system is not only able to determine how things might develop, but also to compute a recommendation in the light of the desired outcome. As a result, the system is able to judge the different developments.

According to an embodiment, a diachronic analysis may be performed in order to predict expected future TKGs when a set of possible actions are executed at certain points in time. To this end, the recommender system may include a diachronic analyzer configured to receive as input a predefined list of actions, where each action has an associated score that describes the cost of implementing the respective action together with the results from the graph analyzer, in particular in form of classified Knowledge Graphs and a corresponding weight matrix. The diachronic analyzer may add, for each action of the list of actions and possible combinations thereof, the respective action or combination of actions to the respective present Knowledge Graph and the corresponding future Knowledge Graph. Based thereupon and by using the prediction component, the further developments of both Knowledge Graphs may be predicted.

According to an embodiment, the recommender system comprises an evaluation module that is configured to compute recommendations of actions (together with their recommended time of execution) based on the graph classification results received from the diachronic analyser. In addition, it may also take into account a model uncertainty of the graph classification results, a difference to alternative scenarios and/or a respective cost associated with each of the actions. Specifically, the evaluation module may analyse the received Knowledge Graphs and their corresponding weight matrices together with confidence scores for the respective actions and, based on the differences between the weight matrices and the confidence scores, determine which of the actions is most effective to turn a Knowledge Graph into the state of interest.

According to an embodiment, the recommender system comprises an explanation module that receives a ranked list of actions from the evaluation module, enables interaction with a user of the recommender system, and transforms the ranked list of actions into configurable representations, including text, images, and/or voice representations.

Embodiments of the present invention can be used in many applications where recommender systems may be required. For example, it can be used in any instance where sensor networks are available to observe the domain, such as in the public services, public safety or biomedical domains. For instance, according to a specific example, the invention may be used for performing traffic control in a smart city, where the information may be collected from a sensor network of a smart city. According to alternative examples, the invention may be used in healthcare scenarios for patient treatment, where information may be collected from a monitoring system that monitors vital parameters of a patient, in public safety applications, e.g. for law enforcement, where the required information may be collected by observing online communications of a person in an online community, or for product placement in retail solutions, where the required information may be collected from a sensor system of a retail store observing customers' behaviour. As will be appreciated by those skilled in the art, the above list is only of exemplary character and a large number of further application scenarios can be envisioned.

There are several ways how to design and further develop the teaching of the present invention in an advantageous way. To this end it is to be referred to the dependent claims on the one hand and to the following explanation of preferred embodiments of the invention by way of example, illustrated by the FIGURE on the other hand. In connection with the explanation of the preferred embodiments of the invention by the aid of the FIGURE, generally preferred embodiments and further developments of the teaching will be explained.

The only FIGURE illustrates an overview system architecture 100 for deploying a recommender system according to an embodiment of the invention. The recommender system runs on a platform where a database server stores information related to different items, situations and users (e.g., images, texts, audio/video recordings, data, etc.).

The system architecture 100 provides several interfaces for enabling interactions with a user 102 of the recommender system.

According to the illustrated embodiment, the system includes multiple modules:
An AI unit 104, to obtain information from a domain of interest 106 and to store the obtained information in a Temporal Knowledge Graph (TKG),
a Future-Link-Entity prediction module 108, to predict future TKGs,
a Graph Analyzer module 110 to classify and to compare the KGs,
a Diachronic Analyzer module 112, which simulates possible future scenarios and analyses how certain actions would influence the KGs (present and future),
an evaluation module 114 to evaluate the created future scenario KGs and to provide a ranked list of actions, effects and outcomes, and
an explanation module 116 to provide those actions as well as explanations to the user 102, and to interact with the user 102.

In the following, the configuration and functionality of each of the respective modules is described in detail.

According to some embodiments, the AI unit 104 dynamically interacts with the domain of interest 106 in order to continuously gain more information. Depending on the domain of interest 106, which may be organized as a knowledge graph of interest and which may be, e.g., an online community, a smart city, a patient or the like, this may be accomplished by respectively adapted interactions, for instance by observing data from a monitoring system or by reading data from a sensor network. Depending on the implementation, the AI unit 104 can also take into account instructions from the user 102. According to an embodiment, the interaction between the AI unit 104 and the domain of interest 106 may be fully automated. Advantageously, in such case the AI Unit 104 may be implemented, e.g., in form of a bot.

Everything which is logged/observed (e.g. through a sensor network) by the AI unit 104 is transformed into a Knowledge Graph (KG), i.e., a set of triples. In this regard, the AI unit 104 may be seen as an information extraction pipeline that (continuously or periodically) extracts new triples. This set of triples represents entities and objects, relation between them, and corresponding attributes. According to some embodiments, the AI unit 104 may be configured to store the information found in a Temporal Knowledge Graph (TKG), i.e. a KG that also contains temporal facts indicating relationships among entities and objects at different times. Embodiments of the invention combine (i.e. take into account) the time-dependent triples with a list of actions and a scope of desired outcomes (i.e. States of Interest) to compute recommendations, as will be described in more detail below. It should be noted that the success of the recommendations depends on how well it is possible to observe/read the domain of interest 106 and convert it into a Knowledge Graph, and how well the graphs are classified by the Graph Analyzer 110 (see below). Hence, the richness of the knowledge base and the neural network architecture of the individual components are important aspects and, as a consequence, the recommender system will have to be carefully tuned for each particular use case.

A prediction component, namely the Future-Link-Entity prediction module 108 (hereinafter briefly denoted FLE prediction module), is configured to take from the AI unit 104 i) the newest KG (hereinafter denoted KGₜ, where t is a timestamp that refers to the present), ii) prediction results from the past, and iii) known knowledge from the database as input. The prediction results from the past (as mentioned at ii)) may be provided as a set of pairs of KGs (G={(KGₜ^{t1}, KGₜ₊ₓ^{t1}) . . . (KGₜ^{tn}, KGₜ₊ₓ^{tn})}), where x describes a step in time, t+x is a timestamp that refers to the future (t+x>t), t1 . . . tn are timestamps that indicated when the respective pair was processed. According to embodiments, the “known knowledge” (as mentioned at iii)) may include everything, which is (publicly) available, e.g., Wikipedia articles or information extracted from services like OpenStreetMap. For instance, in the context of smart city applications, the FLE prediction module may consider the “known knowledge” from, e.g., OpenStreetMap to learn and understand, for example, which routes are suitable for redirecting traffic. On the other hand, in the context of healthcare applications, the FLE prediction module may consider the “known knowledge” from, e.g., Wikipedia to learn and understand, for instance, how certain drugs are constituted. (Based on the chemical attributes, the recommender system can decide whether it should recommend the combination of certain drugs.)

The FLE prediction module 108 may be implemented in form of a neural network whose weights may be trained with stochastic gradient descent (SGD) using the known knowledge. The FLE prediction module 108 produces a new Knowledge Graph (KGₜ₊ₓ) which represents the future of the input KG (KGₜ). In this context, it should be noted that in the new Knowledge Graph the attributes of the entities and objects may have changed and that it might have new edges and nodes, while preexisting nodes and edges can vanish. According to embodiments, the operation of the FLE prediction module 108 may follow the principles described in Garcia-Duran, Alberto, and Mathias Niepert. “Kblrn: End-to-end learning of knowledge base representations with latent, relational, and numerical features.”, in Proceedings of the 34^{th} Conference on Uncertainty in Artificial Intelligence, 2018, which is hereby incorporated herein in its entirety by way of reference.

The graph analyzer module 110 communicates with the FLE prediction module 108 as well as with a diachronic analyzer module 112. Further, the graph analyzer module 110 enables interaction with a user 102 of the recommender system by means of a corresponding interface. The graph analyzer module 110 receives as input the input and output of the FLE prediction module 108, i.e. KGₜ and KGₜ₊ₓ, as well as a predefined list of interest. This list of interest, which may be user-defined, may be provided in form of a list C={c₁ . . . cₙ}, where cᵢ denote different classification labels.

The graphs that were analyzed in the past (i.e. G={(KGₜ^{t1}, KGₜ₊ₓ^{t1}) . . . (KGₜ^{tn}, KGₜ₊ₓ^{tn})}, as introduced above, with the corresponding weight matrices, and classification results) can be used as training data. The GA module 110 may be implemented as a neural network whose weights are trained with stochastic gradient descent (SGD) using the known knowledge. The GA module 110 classifies the initial input and the output of the FLE prediction module 108, i.e., KGₜ and KGₜ₊ₓ. According to embodiments, the operation of the GA module 110 may follow the principles described in Mathias Niepert, Mohamed Ahmed, and Konstantin Kutzkov. “Learning convolutional neural networks for graphs.”, in International conference on machine learning, pp. 2014-2023. 2016, which is hereby incorporated herein in its entirety by way of reference.

According to an embodiment, the GA module 110 may further compute the difference between the respective two graphs via a weight matrix (w_{|KG_(t)-KG_(t+x)|}), which reflects the actual changes between the two graphs. As a result, the GA module 110 returns the weight matrix and for each value in the list of interest c∈C (i.e., for each possible class) a confidence score P for the respective KG (i.e. classification result m={(c, k, P(c|k))|∀c∈C, ∀k∈{KGₜ, KGₜ₊ₓ}}).

The diachronic analyzer (DA) module 112 communicates with the FLE prediction module 108, the graph analyzer module 110 as well as with the evaluation module 114. Further, the diachronic analyzer module 112 enables interaction with a user 102 of the recommender system by means of a corresponding interface.

According to the illustrated embodiment, the diachronic analyzer module 112 receives as input i) a predefined list of actions (A={(a₁,v₁), . . . , (aₙ, vₙ)}), where aₓ denotes an action and vₓ a score that describes the cost of implementing the action, and ii) the results from the GA module 110, i.e., the classified KGs and the corresponding weight matrix. For each possible action and combination of these, the DA module 112 adds the actions (in the form of triples) to both KGs (i.e. KGₜ and KGₜ₊ₓ) and subsequently predicts the further development for both (with the help of the FLE prediction module 108).

For a particular action a E A (or combination of actions), this results in six KGs and the corresponding weight matrices; hence, a) the initial one (namely KGₜ), b) the predicted future of a) (namely KGₜ₊ₓ), c) the initial one with the added action (denoted KG^{a}ₜ), d) the predicted future of c) (denoted KG^{a}ₜ₊ₓ), e) the predicted future of a) with the added action (denoted KGₜ₊ₓ^{a}), and f) the predicted future of e) (denoted KG_{t+x+y}^{a}, where y is a step in time, i.e., t+x+y is a timestamp in the future where t+x+y>t+x).

In other words, according to an embodiment of the invention, the recommender system produces 1) a pair of KGs, which reflects the status quo and the future development without intervention/action, 2) a pair of KGs, which reflects (simulates) that a particular action is performed now (i.e. at present, denoted with timestamp t) and the respective future development, and 3) a pair of KGs, where the respective action is performed in the future (denoted with timestamp t+x) and the respective future development. Consequently, having defined one possible action, the result is a prediction for three pairs of KGs. It should be noted that the meaningfulness of the prediction results from the information about time, time shift and action take.

All of these KGs are analyzed by the GA module 110, i.e., the corresponding weight matrix w and the classification results m are computed, as described already above. Across all actions, the comparison of the corresponding matrices allows to extract the strength of the influence of the action. Hence, by comparing the corresponding matrices it is possible to identify the action which influences the future the most in respect of the interest of the user 102. As a result, the system provides a time-dependent recommendation, i.e., when to act (i.e., now, in the future, never), what should be done (i.e. which action), and how the action influences the development of the KG (i.e., how the future is changed).

It should be noted that for clarity and ease of explanation, the functionality of the DA module 112 has been described for the case of a single time step only. However, as will be appreciated by those skilled in the art, the functionality can be likewise expanded to two or even more time steps. By taking different time steps into account, the space of recommendations is broadened and, as a result, the recommender system can deliver more persuasive recommendations.

According to the illustrated embodiment, the output of the DA module 112 is forwarded to the evaluation module 114. The evaluation module 114, which may be implemented in form of a neural network, is configured to analyze the three (or more, depending on the number of time steps applied by the DA module 112) pairs of KGs and the corresponding weight matrices together with the confidence scores for the respective actions.

Further, according to an embodiment, the evaluation module 114 may also compare the KGs across the pairs and compute the respective weight matrix. Based on the differences between the weight matrices and the confidence scores, the evaluation module 114 computes which action is most effective to turn the KG into the State of Interest. The evaluation module 114 may take into account that an action aₖ might be more effective than another action ah, but action ah might be easier to implement. In this context, it may be provided that the level of complexity of implementing an action is specified by the user 102 through a score, which may be provided together with the list of actions.

The result, i.e. the output of the evaluation module 114, is a ranked list of actions, the corresponding effect (i.e. how the KG changes), and the outcome (i.e. the State of Interest). By using a Temporal Knowledge Graph (TKG) based concept to model the (time-dependent) entities, relations, and attributes, the computed recommendations are highly reliable.

According to some embodiments, the output of the evaluation module 114 is passed to the explanation module 116, which transforms the input into an appropriate representation (e.g. text, images, voice, or the like). In other words, the explanation module 116 provides an explanation to the (human) user 102 which actions have the highest effect, but also why and how it effects the development. According to embodiments, the operation of the explanation module 116 may follow the gradient rollback approach described in Carolin Lawrence, Timo Sztyler, and Mathias Niepert. “Explaining Neural Matrix Factorization with Gradient Rollback.” arXiv preprint arXiv:2010.05516 (2020), which is hereby incorporated herein in its entirety by way of reference.

According to an embodiment, the recommender system may be designed in a way that the user can interact with the explanation module 116, for instance, ask questions for clarifications, which may result in an adaptation of the representation of the explanation. Based on the findings, the user 102 can adapt the DA module 112, i.e., modify the list of possible actions, and/or instruct the actual AI unit 104 that monitors and interacts with the domain of interests 106. Alternatively, instead of interactions by the user 102, it may be provided that the system directly adapts or modifies the AI unit 104 or other system components, preferably in an automated way.

Embodiments of the present invention provide a method for executing a recommender system for time-dependent actions based on Temporal Knowledge Graphs that comprises the following steps:
1. Automated collection of information in a domain of interest 106 through observation and interaction and organization of this information in a Temporal Knowledge Graph (TKG).
2. Prediction, by Future-Link-Entity Prediction module 108, of future links and entities for a given TKG. The Future-Link-Entity Prediction module 108 may include a neural network that is given a set of pairs (present and future) of TKGs as training data and the actual TKG for which the future development should be computed. The output is a TKG which reflects the future of the input TKG.
3. Characterization, by Graph Analyzer 110, of given and expected situations by analyzing the TKG for all time steps. The Graph Analyzer 110 may include a neural network that is given a set of classified pairs (present and future) of TKGs as training data, the States of Interest defined by a human (class labels), and the TKG which needs to be classified. The outcome is a confidence score for each State of Interest and a weight matrix that reflects the difference between the present TKG and the respective future TKG.
4. Prediction, by Diachronic Analyzer 112, of expected future TKG when the set of possible actions are executed at certain points in time. The input for Diachronic Analyzer 112 may be the output of 2) and 3) and a set of actions defined by a human. Using 2) and 3), the output is a set of alternative timelines represented by TKGs.
5. Provision, by Evaluation Module 114, of a list of recommended actions, their expected effect and outcome, by evaluation of the set of TKGs for multiple time steps and actions. The Evaluation Module 114 may include a neural network that is given the output of 4). The output is a ranked list of actions, their effect (how they change the TKG) and the outcome (i.e. the State of Interest).

Embodiments of the present invention achieve the following advantages compared to available prior art solutions:
1. Enriched information representation compared to AI-based (i.e. non KG-based) recommender systems by incorporation of all available data, data modalities, and dimensions.
Relational Data: Using a Knowledge Graph representation enables usage of relational data and thus consideration of knowledge about relationships.
Multi-Dimensionality: The recommender system according to embodiments of the invention is able to incorporate various dimensions, e.g. attributed entities.
Multi-Modality: The recommender system according to embodiments of the invention is able incorporate multi-modal data through embedding the information for getting a vector representation. This enables the use of all available data sources (e.g. videos, images, recordings) and thus increases adaptiveness to different embodiments.
2. More Realistic Representation of dynamic complex real-life scenarios compared to Graph-based recommender systems by consideration of time aspect in two ways.
Dynamicity: The recommender system according to embodiments of the invention is able to (a) update as soon as new information is available, and it can (b) predict appearance of previously unseen nodes and edges, as well as the disappearance of nodes and edges. This leads to the advantages compared to other graph-based recommender systems of (a) being able to work in real-time environments and being able to keep and up-to-date representation even if the situation changes; and (b) being able to predict more realistically future knowledge graphs.
Time aspect of possible actions: Embodiment of the invention consider that actions can take place at different time steps, that the outcome will change over time steps (the future in t+1 is not the same as the future in t+2), and that the same action, taking place at different time steps can lead to different outcomes. This leads to the advantages of more realistic predictions compared to other recommender systems.
3. Overarching decision making due to Evaluation Module 114
The Evaluation Module 114 computes recommendation of actions not only based on the graph classification result, but also takes into account the model uncertainty of the classification result and the difference to alternative scenarios, as well as the cost of each actions. Thus, embodiments of the invention can provide more sophisticated and well-reasoned action recommendations compared to other recommender system who compute recommendations based on less decision criteria.
4. Higher Acceptance and Accountability of Recommendations compared to other graphs-based recommender system through explainability
Embodiments of the invention provide substantiated action recommendations, by providing explanations on the decisions to the user. This leads to higher acceptance of recommendations by human users due to transparency, as well as to easier traceability of potential errors.

The recommender system according to embodiments of the present invention may be suitably applied in connection with traffic control in an intelligent traffic routing system of a smart city. Smart cities are characterized in that they operate a sensor network comprising a huge number of sensors that altogether provide a rich set of information. This information may include, for instance, Information on the satisfaction of the citizen, crimes, waste, traffic issues, control of water and electricity. In this context, the recommender system according to embodiments of the invention is able to provide recommendations to optimize and redirect the traffic flow in respect of pollution, scene of crime, traffic jams, and other events. Hence, the recommender system gathers all kind of information available through the sensor network as a Temporal Knowledge Graph and computes and predicts the future development/trend. Taking the environment parameters into account, i.e. the States of Interest, the system may be configured to provide a ranked list of recommendations how to redirect the traffic (action), when to redirect it (time), and the actual effect resulting from the respective intervention. The output of the recommender system can be used/evaluated by a human to adjust the intelligent traffic routing system. Alternatively, these systems can be directly adapted in a self-adjusting way, i.e. without any human in the loop.

According to an alternative application scenario, a recommender system according to embodiments of the invention may be applied in the healthcare sector, in particular for improved patient treatment. While observing the vital parameter of a patient, e.g. in a hospital, the recommender system according to embodiments of the invention can provide treatment recommendations, e.g., to optimize the condition of the patient. In this context, the recommender system may analyze the current state of the patient and predict the future development of the respective vital parameters, e.g., of the health condition/status. Taking a state of interest into account (e.g. less pain or a lower blood pressures, as specified by, e.g., the attending physician), the recommender system may provide a ranked list of recommendations how to treat the patient (action), when to treat him/her (time), and the actual effect of the treatment. For instance, the time of a certain treatment might be crucial in respect of the dose where the effect can exposure unwanted side-effects. The treatment recommendation can be evaluated and applied by a human, e.g., by adjusting the medical devices (e.g. ventilator or the amount of morphine). Alternatively, these devices can be directly adapted in a self-adjusting way, i.e. without any human in the loop.

According to still another application scenario, a recommender system according to embodiments of the invention may be applied in the public safety sector or, in particular for law enforcement. For instance, in order to detect an alteration (e.g. radicalization) of a person in an (online) community, a recommender system according to embodiments of the invention can observe (online) communications to understand their topics, domains, and individual opinions. In terms of online communities, this might be in a forum or any social media page where non-online communities could be traced through a monitoring system (e.g. cameras and microphones). For both cases, an AI unit may record and convert the happenings. Through the analysis of the Temporal Knowledge Graph, the recommender system may detect (a shift towards) and predict an alteration (e.g. radicalization) in future time steps. Further, the recommender system may identify the reasons and factors for the alteration which are used to compute effective actions. Depending on the scenario, the output are recommendations for actions, which support or counteract the alteration. For instance, based on the output, a police authority can adjust the chat bot in the online community or adapt the monitoring system in terms of what should be record or displayed.

According to a further application scenario, a recommender system according to embodiments of the invention may be applied in connection with product placement in smart retail scenarios. For instance, in a smart retail store that is equipped with cameras to track the customers, a recommender system according to embodiments of the invention can use the sensor network of the shop to observe the behavior of the customers. Thus, the system can analyze the buying behavior of the customer in respect of product placement but also analyze when products run out. Taking the state of interest into account (e.g. which products should be sold with priority, or when to actually restock certain products) the system can compute recommendations how to arrange products or when to restock them (e.g. considering perishable products). This also includes time-dependent product arrangement recommendations. Based on the output, an employee could adjust the product placement robot in terms of work instructions. Alternatively, the robot can be directly adjusted without any human in the loop (e.g. in an autonomous retail store).

Many modifications and other embodiments of the invention set forth herein will come to mind to the one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1: A method for providing recommendations to users based on a state of interest, the method comprising:
organizing domain of interest information in an initial temporal knowledge graph (KGₜ), wherein t is a timestamp that refers to a present point in time;
predicting, for at least one future point in time (t+x), future entities, future links between entities and/or future attributes of entities for the initial knowledge graph (KGₜ) and producing at least one new knowledge graph (KGₜ₊ₓ) based on the predictions;
simulating situations resulting from the execution of a particular action or a combination of actions at certain points in time and predicting expected temporal knowledge graphs for the simulated situations;
classifying the knowledge graphs produced for the respective points in time and for the simulated situations based on the state of interest; and
providing, based on the classification result, a ranked list of recommended actions.

2: The method according to claim 1, wherein the predicting future entities, future links between entities and/or future attributes of entities for the initial knowledge graph is performed by a neural network, wherein weights of the neural network are trained with stochastic gradient descent (SGD) using past prediction results as training data.

3: The method according to claim 1, further comprising:
characterizing present and expected situations by analyzing the temporal knowledge graphs for the different points in time by means of a graph analyzer.

4: The method according to claim 3, wherein the graph analyzer comprises a neural network that uses a set of classified pairs of present and future temporal knowledge graphs as training data and that classifies a temporal knowledge graph based on a state of interest that is defined by a set of classification labels by determining a confidence score for each state of interest.

5: The method according to claim 3, further comprising
calculating, by the graph analyzer, a weight matrix that reflects the difference between a respective present knowledge graph and a corresponding future knowledge graph.

6: The method according to claim 5, further comprising:
receiving, by a diachronic analyzer, a predefined list of actions, wherein each action has an associated score that describes a cost of implementing the respective action, together with results from the graph analyzer;
adding, by the diachronic analyzer, for each action of the list of actions and possible combination thereof, the respective action or combination of actions to the respective present knowledge graph and the corresponding future knowledge graph and predicting the further developments of both knowledge graphs.

7: The method according to claim 6, further comprising:
receiving, by an evaluation module, results from the diachronic analyser;
analysing, by the evaluation module, the received knowledge graphs and their corresponding weight matrices together with confidence scores for the respective actions; and
determining, by the evaluation module based on the differences between the weight matrices and the confidence scores, which of the actions is most effective to turn a knowledge graph into the state of interest.

8: The method according to claim 1, wherein the domain of interest information is collected and/or acquired through observation and/or interaction from:
a sensor network of a smart city, and/or
a monitoring system that monitors vital parameters of a patient, and/or by observing online communications of a person in an online community, and/or a sensor system of a retail store observing behaviour of customers.

9: A recommender system for providing recommendations to users based on a state of interest, the system comprising:
an artificial intelligence (AI) unit configured to organize domain of interest information in an initial temporal knowledge graph (KGₜ), where t is a timestamp that refers to a present point in time;
a prediction component configured to predict, for at least one future point in time (t+x), future entities, future links between entities and/or future attributes of entities for the initial knowledge graph (KGₜ) and to produce at least one new knowledge graph (KGₜ₊ₓ) based on the predictions;
a diachronic analyzer configured to simulate situations resulting from the execution of a particular action or a combination of actions at certain points in time and to predict expected temporal knowledge graphs for the simulated situations;
a graph analyzer configured to classify the knowledge graphs produced for the respective points in time and for the simulated situations based on the state of interest; and
an evaluation module configured to provide, based on the classification result, a ranked list of recommended actions.

10: The system according to claim 9, wherein the prediction component comprises a neural network, wherein weights of the neural network are trained with stochastic gradient descent, SGD, using past prediction results as training data.

11: The system according to claim 9, wherein the graph analyzer is configured to characterize present and expected situations by analyzing the temporal knowledge graphs for the different points in time.

12: The system according to claim 9, wherein the diachronic analyzer is configured to:
receive as input a predefined list of actions, where each action has an associated score that describes a cost of implementing the respective action;
receive results from the graph analyzer;
adding, for each action of the list of actions and possible combinations thereof, the respective action or combination of actions to the respective present knowledge graph and the corresponding future knowledge graph; and
predicting, by using the prediction component, the further developments of both knowledge graphs.

13: The system according to claim 9, wherein the evaluation module is configured to
receive results from the diachronic analyser;
analyse the received knowledge graphs and their corresponding weight matrices together with confidence scores for the respective actions; and
to determine, based on the differences between the weight matrices and the confidence scores, which of the actions is most effective to turn a knowledge graph into the state of interest.

14: The system according to claim 9, wherein the evaluation module is configured to compute recommendation of actions based on graph classification results and by additionally taking into account a model uncertainty of the graph classification results, a difference to alternative scenarios and/or a respective cost associated with each of the actions.

15: The system according to claim 9, further comprising an explanation module that is configured to:
receive a ranked list of actions from the evaluation module;
interact with a user; and
transform the ranked list of actions into configurable representations, including text, images, and/or voice representations.

16: The method according to claim 8, wherein vital parameters of the patient comprise pain levels and/or blood pressure levels, and the method further comprises:
providing treatment recommendations to optimize a condition of the patient.

17: The method according to claim 6, wherein the results from the graph analyzer are presented in form of classified knowledge graphs and a corresponding weight matrix.

18: The method according to claim 12, wherein the results from the graph analyzer are presented in form of classified knowledge graphs and a corresponding weight matrix.
